(19) United States
(12) Patent Application Publication
(10) Pub. No.: US 2021/0169410 A1
(43) Pub. Date: Jun. 10, 2021

(54) WEARABLE DEVICE TO SCREEN OPIOID INTOXICATION

(71) Applicants: Ghassan S. Kassab, La Jolla, CA (US); Ali Dabiri, San Diego, CA (US); Ludwig J. Weimann, San Diego, CA (US)

(72) Inventors: Ghassan S. Kassab, La Jolla, CA (US); Ali Dabiri, San Diego, CA (US); Ludwig J. Weimann, San Diego, CA (US)

(21) Appl. No.: 17/116,566

(22) Filed: Dec. 9, 2020

Related U.S. Application Data
(60) Provisional application No. 62/945,614, filed on Dec. 9, 2019.

(51) Int. Cl. A61B 5/00; A61B 5/1473; A61B 5/145

(52) U.S. Cl. CPC A61B 5/4845; A61B 5/685; A61B 5/14735; A61B 5/14517; A61B 5/6833; A61B 5/68335; A61B 5/14514

(57) ABSTRACT

A microneedle device comprising a membrane having an adhesive thereon and a microneedle substrate adhered to the membrane via the adhesive. The microneedle substrate may have a plurality of microneedles coupled thereto, or the microneedle substrate can comprise the plurality of microneedles. The microneedle device can be mated with a reagent container and the microneedles aligned with wells on the reagent container said wells configured to hold reagents thus comprising a system for detecting opioids or other drugs. In another embodiment, the device comprises a sweat-absorbent swatch adhered to a membrane. This embodiment can be mated to a screening pad comprising blisters of reagents on the base layer of the screening pad. Upon mating, a needle device can be used to pierce the blisters such that the reagents are released and react with the sweat absorbent swatch to indicate the presence of opioids or other drugs.

### PRIORITY

The present patent application is related to, and claims the priority benefit of, U.S. Provisional Patent Application Ser. No. 62/945,614, filed on Dec. 9, 2019, the contents of which are hereby incorporated by reference in their entirety into this disclosure.

### BACKGROUND

About two million Americans are addicted to opioid drugs, including prescription pain medicines, heroin and fentanyl or one of its analogues. Many millions more misuse opioids, taking opioid medications longer or in higher doses than prescribed. These statistics are staggering, and the tragic effects of the opioid crisis do not stop there but extend to our entire nation.

The negative impact of these drugs is even greater when used by public first responders, pilots, firefighters, soldiers, and individuals with public responsibilities. Increased overdose and misuse of opioids in the United States (US) makes it more important than ever to have full capability to detect drugs that can impair judgment in subjects responsible for public safety. Between 1999 and 2016, more than 630,000 people died from a drug overdose in the US. The current epidemic of drug overdoses began in the 1990s with overdose deaths involving prescription opioids, driven by dramatic increases in prescribing of opioids for chronic pain. In 2010, rapid increases in overdose deaths involving heroin marked the second wave of opioid overdose deaths. The third wave began in 2013, when overdose deaths involving synthetic opioids, particularly those involving illicitly manufactured fentanyl, began to increase significantly. In addition to deaths, nonfatal overdoses from both prescription and illicit drugs are responsible for increasing emergency department visits and hospital admissions. Roughly 118,000 people died as a result of opioid use disorders in 2015.

Opioids are a drug class that includes heroin, synthetic opioids such as fentanyl (and analogues), and pain relievers such as oxycodone, hydrocodone, codeine, morphine, and others. Side effects of opioids include sedation, nausea, respiratory depression, and euphoria. Fentanyl and analogues have rapid onset of symptoms and vary in duration of action. These drugs are 50-100 times more potent than morphine, which predispose individuals to quantities leading to accidental life-threatening exposure. Because of the risks associated with the low dose required for rapid onset of impairment, there is significant interest in real-time detection of exposure and diagnosis of intoxication at the point-of-need through a wearable medical device.

### Sweat Transdermal Patches

Transdermal patches are now widely used as cosmetic, topical, and transdermal delivery systems. These patches are the result of great progress in skin science, technology, and expertise developed through trial and error, clinical observation, and evidence-based studies that date back to the first existing human records. The advantage to using a sweat transdermal patch is the long testing window. Although standard urine-based test strips may be better for immediate results, they only detect drugs that have been metabolized. Sweat patches, however, also detect the parent drug. The longer testing window helps when detecting the most common drugs, such as marijuana, cocaine, methamphetamines, lysergic acid diethylamide (LSD), and heroin, which generally stay in the system of occasional users for about five days.

Urine testing can often miss the detection of drugs as they can only detect the metabolite. Commercially available sweat patches, on the other hand, can detect the parent drug. The variation between individuals in the amount of sweat they excrete has caused difficulty to construct a universal sweat collection device. Earlier attempts to test for the presence of specific substances in sweat have used patches that occlude the skin causing side effects, such as skin irritation, alteration of both the steady-state pH of the skin, and colonization of skin bacteria. Newer, nonocclusive patches use a transparent film that allows oxygen, carbon dioxide, and water vapor to diffuse, while trapping the necessary traces of drug substance excreted in sweat. The newer patch has many benefits including high subject acceptability, low incidence of allergic reactions to the patch adhesive, and ability to monitor drug intake for a period of several weeks with a single patch. Several studies have also found that the patch is resistant to inconspicuous tampering. It has also reported that no special precautions were needed to wear the patch for several days, except to avoid excessive towel rubbing after bathing. Some disadvantages include high inter subject variability, possibility of environmental contamination of the patch before application or after removal, and accidental removal during the monitoring period. In addition, it was reported that the cost of patch testing, based on the panel of drugs tested, was five times that of urine tests. Validation of results from sweat patches, most of which use urine testing as the “gold standard,” have been controversial. It has been reported that good inter-patch reliability and concurrent validity with urine tests when testing for methadone, opiates, and morphine, while tests for cocaine revealed only a moderate level of agreement. In a noted study specifically designed to find possible sources of contamination, it was found that precautionary methods, including cleansing the skin before patch application, are not completely reliable in preventing contamination from the environment. Chawarski et al. evaluated the utility of sweat testing for monitoring of drug use in outpatient clinical settings and compared sweat toxicology with urine toxicology and self-reported drug use during a randomized clinical trial of the efficacy of buprenorphine for treatment of opioid dependence in primary care settings. All study participants were opiate dependent, treatment-seeking volunteers. The findings suggest limited utility of sweat patch testing in outpatient settings. The commercially available transdermal patches need to be transported to a diagnostic laboratory after removal for drug detection.

### Interstitial Body Fluid Transdermal Microneedles

Microneedle arrays are minimally invasive devices that can be used to bypass the stratum corneum barrier and thus accessing the skin microcirculation and achieving systemic delivery by the transdermal route for drug delivery. Microneedles (MN) (hundreds of microns in length up to 1000 MNcm⁻²) with diverse geometries have been produced from silicon, metal, and polymers using various microfabrication techniques. MNs have been prepared using chemical isotropic etching, injection molding, reactive ion etching, surface/bulk micromachining, micro-molding and lithography-electroforming-replication. MNs are applied to the skin surface and pierce the epidermis (devoid of nociceptors) painlessly without skin infection, creating microscopic holes through which drugs diffuse to the dermal microcirculation. MNs can be made long enough to penetrate to the dermis layer but are typically short and narrow enough to avoid stimulation of dermal nerves and puncture dermal blood vessels. MNs are classified as solid, hollow, and polymeric depending on the application. Solid MNs puncture skin prior to application of a drug-loaded patch or are pre-coated with drug prior to insertion. Hollow bore microneedles allow diffusion or pressure-driven flow of drugs through a central lumen. The polymeric MNs are either of dissolved type or hydrogel-forming. The dissolved MNs release their drug payload as they dissolve in the skin layers and are generally a biocompatible polymer. The skin insertion of the array is followed by dissolution of the MNs tips upon contact with skin interstitial fluid. The drug is then released over time. The hydrogel-forming MNs take up interstitial body fluids (IBL) from the tissue, inducing diffusion of the drug located in a patch through the swollen micro-projections. The amount of swelling can be controlled by adding different agents. Hydrogel-forming MNs are removed intact from skin, leaving no measurable polymer residue behind. They cannot be reused since there is a potential of getting softer. MN polymers are drawing increasing attentions because of their excellent biocompatibility, biodegradability, low toxicity and strength/toughness. They are easy to fabricate and cost-effective.

### BRIEF SUMMARY

### Transdermal Patches

A primary objective of the methods and apparatuses/devices of the present disclosure is to provide new systems and technique to screen opioids more readily and inexpensively than the current systems via transdermal patches. The devices disclosed herein have the following features/benefits: 1) non-invasive, 2) real-time response after removal (Point of Care), 3) passive device which does not need the cooperation of the subject, 4) ability of patch to screen the presence of all opioids of interest at the same time for a desired period of residence time with a single patch, 5) bio-material compatibility, 6) ease of manufacturing, 7) low cost, 8) long shelf life, 9) ease of application/removal, 10) wearable and easy to handle in any application setting, 11) no skin side effects like irritation or allergic reactions to the patch adhesive, 12) screening with very low false negative, 13) resistant to inconspicuous tampering, 14) possibility of oxygen, carbon dioxide, and water vapor to escape while trapping necessary traces of drug use excreted in sweat, and 15) minimum environmental contamination of patch before application or after removal.

It is imperative to know the order of magnitude of the opioids concentration in sweat to search for method(s) capable to achieve the required Level of Detection (LOD) of opioid agents in the patch screening device. The devices of the present disclosure are optimized to screen the drug agents of interest.

Sweat and sebaceous glands are found in the dermis and are distributed throughout the body disproportionately. The highest concentration of sweat glands resides in the hands, while the forehead contains the densest population of sebaceous glands. Both glands deliver byproducts of drugs to the skin's surface through either sweat or sebum. Drugs are thought to enter the sweat by passive diffusion from the blood stream to the sweat gland. Drugs are also dissolved in sweat on skin's surface after they diffuse through stratum corneum. Despite variation between individuals in sweat production, researchers have successfully used sweat to test for cocaine, opiates, benzodiazepines, and others.

The rate of sweating depends on the skin temperature, which is normally 33° C. The rate of sweating increases by a factor of about four when jogging opposed to resting. This relationship holds even if the skin temperature increases to 36° C. The sweating rate increases by a factor of about four when the skin temperature increases to 36° C. from 33° C. An average person sweats between 0.8 to 1.4 liters per hour (L/hr) during exercise, depending on the type of exercise, metabolic rate, skin surface area, and skin temperature. This rate can increase as high as 4 L/hr. The analysis referenced herein is based on 0.2 L/hr, which is the lower limit of sweating at skin temperature of 33° C. at rest. The skin area is about 1.5 to 2.0 square meters for an average adult that results in the sweat amount of about 0.2 ml for an absorbing area of patch of about 2 cm² in 6 to 8 hours. The longer testing window was selected to help detecting the most common drugs, such as marijuana, cocaine, methamphetamines, LSD, and heroin, which generally stay in the system of occasional users for about five days as previously noted herein.

It has been found that free and total peripheral blood morphine concentrations are consistent with fatal heroin intoxications, averaging 0.16 mg/L and 0.35 mg/L, respectively in cases where acetyl fentanyl or fentanyl were not involved. In the heroin cases with fentanyl present, the average fatal free morphine concentration was 0.040 mg/L, the average total morphine concentration was 0.080 mg/L, and the fatal average fentanyl concentration was 0.012 mg/L. In cases involving only acetyl fentanyl (without heroin), the average fatal acetyl fentanyl concentration was 0.47 mg/L and the average fatal acetyl norfentanyl concentration was 0.053 mg/L. These data indicate that the range of agent concentrations in the blood are 10-350 ng/ml. The opioid concentrations in the sweat may be less than what it would be in blood. The total amount of the opioids collected in the absorbing area of the patch of about 2 cm² size is about 0.2 to 7 ng after 6 to 8 hours of patch residence time for an average active subject assuming the concentration of opioid in the sweat is about 10% of concentration of the same opioid found in the blood. This range will be our design requirement for the appropriate patch screening device. This means that we need to have minimum LOD of about 0.2 ng for the opioids of interest.

### Interstitial Body Fluid Transdermal Microneedles

A sufficient amount of sweat must be absorbed by the patch to generate the desired concentration of the drugs for color change in a reasonable residence time. Microneedles will be considered to generate the color change if the sweat concentration is too low for screening.

Previous studies have shown that 83% of proteins found in serum are also in Interstitial body fluid (IBF), but 50% of proteins in IBF are not in serum, suggesting that Interstitial body fluid may be a source of unique biomarkers as well as biomarkers found in blood. Skin is the most accessible organ and therefore a source of IBF containing biomarkers. Most of skin's IBF is in dermis, which comprises a network of collagen and elastin fibers surrounded by extracellular matrix that limits IBF flow due to binding and tortuosity. It is estimated that ˜70 wt % of human dermis comprising IBF. There are several mechanisms of IBF collection into MN including diffusion, capillary and osmotic actions.

A primary objective of the present method and apparatus is to provide new systems and technique to screen opioids more readily and inexpensively than the current systems via Interstitial body fluid. The device features will be 1) use of polymer microneedles, 2) minimally invasive, 3) Real time response after removal (Point of Care), 4) Passive device which does not need the cooperation of the subject, 5) Ability of microneedle to screen the presence of all opioids of interest at the same time for a desired period of residence time with a single microneedle, 6) Bio-material compatibility, 7) Ease of manufacturing, 8) Low cost, 9) Long shelf life, 10) Ease of application/removal, 11) No skin side effect like irritation or allergic reactions to the microneedle, 12) Screening with very low false negative, 13) Resistant to inconspicuous tampering, and 14) Minimum environmental contamination of microneedle before application or after removal.

Recent progress indicates the possibility of 1-10 μl of IBF within 20 min though MN. As noted above, the sweat amount is ˜0.2 ml for an absorbing area of patch of about 2 cm² in 6-8 hours which corresponds to 10 μl for 20 min. It is a good assumption that the concentration of opioids in the interstitial body fluid is about the same as in the blood concluded from the remarks noted above and the concentration of opioids in the sweat is at most 10% of the corresponding amount in the blood. Therefore, microneedle patches can increase the opioids detectability by a factor of at least 10 for the same patch residence time. This factor increases for those individuals that usually do not sweat.

The present disclosure includes disclosure of a microneedle device, comprising an adhesive layer, and a microneedle substrate adhered to the adhesive layer, and a) wherein the microneedle substrate has a plurality of microneedles coupled thereto, or b) wherein the microneedle substrate further comprises the plurality of microneedles. The microneedle device can be firmly attached to the skin by adhesive layer. The microneedle device, comprising release liner where release liner covers microneedle device during storage and prior to use, so to avoid potential contamination of microneedle device. Release liner is removed before use.

The present disclosure includes disclosure of a microneedle device, comprising a membrane (which, along with an adhesive, can be considered as an “adhesive layer”). And a microneedle substrate adhered thereto (adhered to the membrane, which, along with the adhesive, can be considered to be the adhesive layer), and a) wherein the microneedle substrate has a plurality of microneedles coupled thereto, or b) wherein the microneedle substrate further comprises the plurality of microneedles

The present disclosure includes disclosure of a microneedle device, forming part of a system, the system further comprising at least one of the following a reagent container having wells defined therein, the wells configured to hold reagents, and/or a detection device.

The present disclosure includes disclosure of a method to use a microneedle device, comprising the steps of placing a microneedle device of the present disclosure upon skin of a wearer so to cause at least part of a plurality of microneedles of the microneedle device to enter a dermis of the skin, and removing the microneedle device from the skin after a period of time elapses, said period of time being enough time to permit interstitial body fluid to at least partially coat the plurality of microneedles.

The present disclosure includes disclosure of a method to use a microneedle device, further comprising the step of positioning the plurality of microneedles of the microneedle device into wells of a reagent container so to potentially cause one or more reactions between the interstitial body fluid at least partially coating the plurality of microneedles and reagents within the wells of the reagent container, said one or more reactions resulting in one or more color changes, the one or more color changes indicative of the presence of one or more opioids and/or chemicals related thereto.

The present disclosure includes disclosure of a method to use a microneedle device, wherein the plurality of microneedles are at least partially coated with reagents prior to the step of placing the microneedle device upon the skin of the wearer; and wherein the method further comprises the step of inspecting the plurality of microneedles in attempt to identify one or more color changes thereon, the one or more color changes indicative of the presence of one or more opioids and/or chemicals related thereto.

The present disclosure includes disclosure of a method to use a microneedle device, wherein the plurality of microneedles have lumens defined therethrough, wherein the step of placing the microneedle device upon the skin of the wearer further includes operating a suction device/mechanism coupled to or formed as part of the microneedle device to cause the interstitial body fluid to flow into the lumens of the plurality of microneedles.

The present disclosure includes disclosure of a method to use a microneedle device, further comprising the step of combining the interstitial body fluid with a plurality of reagents so to potentially cause one or more reactions between the interstitial body fluid and the reagents, said one or more reactions resulting in one or more color changes, the one or more color changes indicative of the presence of one or more opioids and/or chemicals related thereto.

The present disclosure includes disclosure of a system, comprising one or more patches, the one or more patches comprising a membrane, a sweat-absorbent swatch, and an adhesive layer; and one or more screening pads, the one or more screening pads comprising a base layer, one or more blisters positioned upon or formed within the base layer, and one or more reagents positioned within one or more of the one or more blisters.

The present disclosure includes disclosure of a system, wherein the one or more patches have a release liner positioned thereon, configured to cover the sweat-absorbent swatch.

The present disclosure includes disclosure of a method to use a system, comprising the steps of placing a patch of the present disclosure upon skin of a wearer, and removing the patch from the skin after a period of time elapses, said period of time being enough time to permit sweat to transfer from the skin to the patch.

The present disclosure includes disclosure of a method, further comprising the step of positioning a screening pad upon a sweat-absorbent swatch of the patch to potentially cause one or more reactions between the sweat on or within the swatch and reagents within the screening pad, said one or more reactions resulting in one or more color changes, the one or more color changes indicative of the presence of one or more opioids and/or chemicals related thereto.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a bottom view of a patch, according to an exemplary embodiment of the present disclosure;

FIG. 2 shows a side view of a patch with a release liner positioned thereon, according to an exemplary embodiment of the present disclosure;

FIG. 3 shows a side view of a patch with a release liner removed therefrom, according to an exemplary embodiment of the present disclosure;

FIG. 4 shows a side view of a patch positioned upon the skin and absorbing sweat therefrom, according to an exemplary embodiment of the present disclosure;

FIG. 5 shows a top view of a screening pad, according to an exemplary embodiment of the present disclosure;

FIG. 6 shows a side view of a screening pad, according to an exemplary embodiment of the present disclosure;

FIG. 7 shows a top view of a screening pad positioned upon a patch, according to an exemplary embodiment of the present disclosure;

FIG. 8 shows a side view of a screening pad positioned upon a patch with the blisters of the screening pad using a needle (or microneedle) device, according to an exemplary embodiment of the present disclosure;

FIG. 9 shows a perspective view of a needle (or microneedle) device, according to an exemplary embodiment of the present disclosure;

FIG. 10 shows a detection device used to detect reacted indications on a patch, according to an exemplary embodiment of the present disclosure; and

FIG. 11 shows a schematic of interstitial fluid collection, such as the collection of IBL using a microneedle patch, and processing said microneedle device to identify one or more reacted indications, according to an exemplary embodiment of the present disclosure;

FIG. 12 shows a microneedle device positioned relative to a reagent container, according to an exemplary embodiment of the present disclosure;

FIG. 13 shows a microneedle device applied to the skin, according to an exemplary embodiment of the present disclosure;

FIG. 14 shows a block diagram of various potential components of a system, according to an exemplary embodiment of the present disclosure; and

FIG. 15 shows a microneedle device having a plurality of hollow needles, the microneedle device coupled to or formed along with a suction device/mechanism, according to an exemplary embodiment of the present disclosure; and

FIG. 16 shows a microneedle device covered by a release liner, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Systems 50 of the present disclosure comprise two parts/portions—a sweat patch 100, also referred to herein as a collection part/portion, and a screening pad 500, also referred to herein as a screening part/portion or a detection part/portion. Screening pad 500 is composed of biomarkers, where it will lay on top of the sweat patch after removal. Screening pad 500 will then be removed from the sweat patch 100 after several seconds for color determination by either the naked eye or using a detection device such as a spectrometer. Sweat patches 100 can be slightly heated to evaporate the residual liquid to increase agent concentrations.

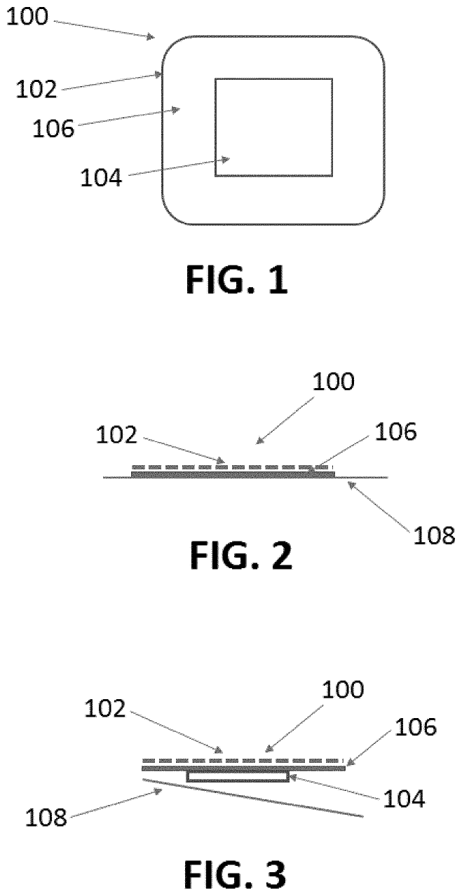

An exemplary patch 100 (also referred to herein as a sweat patch or sweat transdermal patch) of the present disclosure is shown in FIG. 1. As shown therein, sweat patch 100 comprises a membrane 102, a sweat-absorbent swatch 104, and an adhesive layer 106 present upon membrane 102 (an adhesive being applied to membrane 102), whereby adhesive 106 facilitates adhesion of swatch 104 to membrane 102 and adhesion of sweat patch 100 to a wearer's skin.

As shown in FIG. 2, an exemplary sweat patch 100 of the present disclosure can comprise a release liner 108, where release liner 108 covers patch 100 during storage and prior to use, so to avoid potential contamination of swatch 104. Release liner 108 is removed before use, such as shown in FIG. 3, revealing swatch 104. Release liners 108 can have a thickness between 50 to 70 μm, or larger or smaller.

Adhesive layer 106, as noted above, is used so that sweat patch 100 can be firmly attached to the skin of a wearer. Swatch 104, as noted above, is positioned at the center of the adhesive layer 106.

An exemplary adhesive layer 106 of the present disclosure can comprise any number of suitable adhesives, such as bioadhesives (Duro-TAK 387-2510/87-2510 from Henkel, for example) or other materials which is/are mixed with sodium carboxymethyl cellulose (NaCMC) or other materials, resulting in a total adhesive layer 106 thickness of 100 to 150 μm, or thicker or thinner.

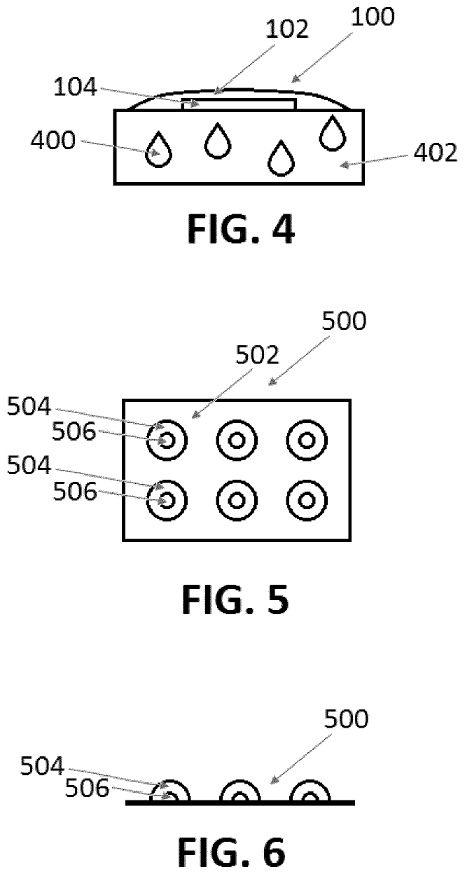

Membranes 102 of the present disclosure essentially exist as a backing film on an opposite side of adhesive layer 106 used to adhere to swatch 104, whereby the configuration of membranes 102 ensure that gases such as oxygen, carbon dioxide, and water vapor can escape into the surrounding external environment. An exemplary membrane 102 of the present disclosure could be 3M, Co Tran TM 9701 Backing Polyurethane Monolayer Film with high moisture vapor transmission rate (MVTR) of 709 g/m²/24 hr, or other suitable membrane 102 materials that permit gas escape as noted herein. The total thickness of the membrane 102 can be 200 to 300 μm, or thicker or thinner. In such a configuration, sweat 400 diffuses from the skin 402 into sweat patch 100 where it is absorbed by swatch 104, such as depicted in FIG. 4.

Manufacture/production of sweat patch 100 can include three steps, as follows:
Step 1: Release liner 108 and adhesive layer 106 are put together, and membrane 102 is added on the outside of the adhesive layer 106.
Step 2: Swatch 104 is placed at the center of the adhesive layer 106 so that swatch 104 can ultimately and directly contact the skin of a wearer of sweat patch 100. In this configuration, the adhesive layer 106 sticks to the skin all around the absorbing swatch 104.
Step 3: Cutting edges and corners of sweat patch 100 to desired dimensions to result in a final sweat patch 100.

Other manufacturing/production methods/steps are also contemplated in the present disclosure, such as whereby membrane 102 is cut to size prior to applying swatch 104 thereto, such as whereby adhesive layer 102 before other portions of sweat patch 100. The end result of any said method or method steps noted above would be a sweat patch 100 configured for use as referenced herein.

In use, rapid evaporation of sweat 400 moisture through membrane 102 constituting the relative top layer of sweat patch 100 can reduce the residence time to few hours.

A top view of an exemplary screening pad of the present disclosure is shown in FIG. 5. As shown therein, screening pad 500 comprises a base layer 502 and a plurality of blisters 504 present thereon. Blisters 504 of the present disclosure are configured to contain/retain one or more reagents 506 therein. In at least one example, each blister 504 would contain one reagent 506. In other examples, the number of reagents 506 (one, two, three, or more) can vary within each blister 504.

An exemplary screening pad 500 of the present disclosure has a plurality of blisters 504, such as two, three, four, five, six (as shown in FIG. 5), seven, eight, or more blisters 504. It is noted that an embodiment of a screening pad 500 of the present disclosure can have only one blister 504 with one or more reagents 506 therein, but such an embodiment would limit the opioid detection to generally one type of opioid. More blisters 504 having reagent(s) 506 therein would permit the detection of several opioids at once, as referenced in further detail herein.

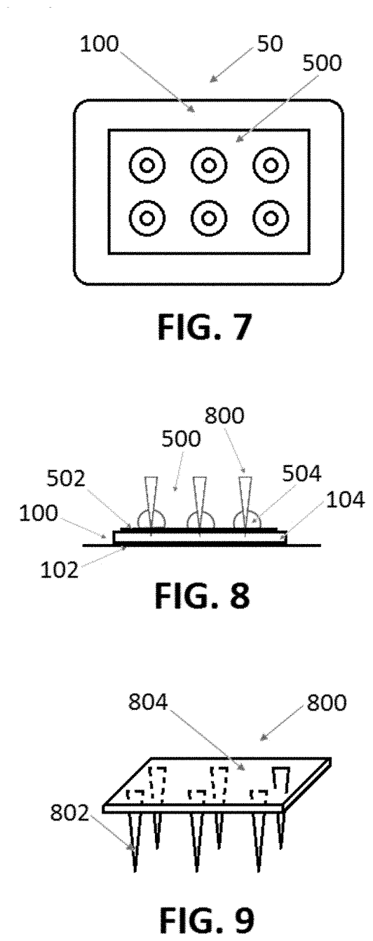

FIG. 6 shows a side view of an exemplary screening pad 500 of the present disclosure, FIG. 7 shows top view of an exemplary screening pad 500 positioned upon a swatch 104 of a patch 100 of the present disclosure, and FIG. 8 shows a side view of an exemplary screening pad 500 positioned upon a swatch 104 of a patch 100 of the present disclosure, whereby a needle device 800 (namely one or more needles 802, including an optional substrate 804) is used to pierce blisters 504 of screening pad 500 to permit reagents 506 to transfer from blisters 504 into swatch 104 of patch 100, so to permit reagents 506 to react with chemicals within the sweat within swatch 104 that are indicative of one or more opioids.

An exemplary screening pad 500 of the present disclosure can contain blisters 504 that each contain one of the reagents shown in Table 1, provided below.

TABLE 1
Screening grid for screening of opioids in human sweat

| | COLOR PRODUCING SENSORS (REAGENTS) | | | | | |
|---|---|---|---|---|---|---|
| | Cobalt- | Ferric | | Ammonium | Ferric | Marquis |
| OPIOID | thiocyanate 1 | Chloride 2 | Eosin Y 3 | Vanadate 4 | Sulphate 5 | Reagent 6 |
| Amphetamine | | | | bluish green | | |
| Meth-amphetamine | | | | dark yellowish green | | |
| Heroin | | | | | | reddish purple |
| Cocaine | greenish blue | | pink | orange yellow | | |
| Fentanyl | | | violet | Dark olive | | |
| Codeine | | | | dark purple | | reddish purple |
| Oxycodone | | | | greenish yellow | | |
| Morphine | | Dark green | | dark reddish brown | | reddish purple |
| Hydrocodone | greenish blue | | | | | |
| Opium | | | | dark brown | brownish purple | |
| Hydromorphone | | | pink | | | |

Table 1 shows a listing of six exemplary reagents for the rapid, real time opioid screening based on the detection grid shown in said table. Eleven exemplary opioids are listed in Table 1 and can be screened by the six reagents. For example, cocaine and hydrocodone can be screened if the reaction of the sweat with cobalt-thiocyanate results in greenish blue color. The raw materials for the reagents are commercially available.

Said reagents 506 may include, but are not limited to, cobalt-thiocyanate, ferric chloride, Eosin Y, ammonium vanadate, ferric sulphate, and Marquis Reagent. Said reagents, as shown in Table 1, are able to detect one or more opioids, including but not limited to amphetamine, methamphetamine, heroin, cocaine, fentanyl, codeine, oxycodone, morphine, hydrocodone, opium, and hydromorphone.

As shown in FIG. 7 and FIG. 8, and after patch 100 has been placed on the skin of a wearer for a time sufficient to collect chemicals within sweat in swatch 104 of patch 100, screening pad 500 is positioned upon patch 104 so that blisters 504 are positioned relatively above swatch 104. A needle device 800, such as a needle device 800 comprising six needles, can puncture blisters 504 one or more at a time or all at once so to let the reagents 506 flow on the surface of the collection part (swatch 104) to generate different colors depending on the type of the opioid contained within or upon swatch 104. Needle devices 800 of the present disclosure can comprise one or more needles 802, such as a plurality of needles 802 effectively coupled to one another via a substrate 804, as shown in FIG. 9.

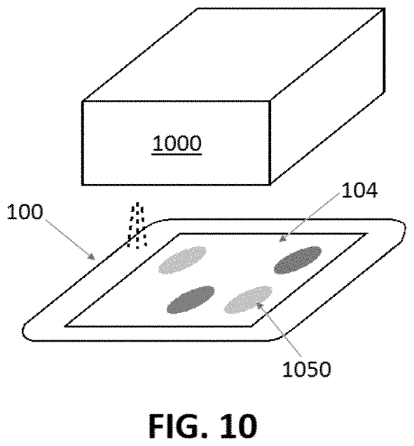

When reagents 506 react with an opioid or a chemical indicative of an opioid present upon or within swatch 104, a color would appear and indicate a reaction between the reagent 506 and the opioid or the chemical indicative of an opioid (referred to herein as a reacted indication 1050, as shown in FIG. 10).

The reacted indications 1050 can potentially be identified visually, and should it be impractical to do so, a detection device 1000 configured to detect reacted indications 1050 can be used, such as being positioned relative to a patch having potential reacted indications 1050 thereon or therein. Such a detection device 1000 could be a portable spectrometer (such as a smartphone spectrometer) or other device, and the detected colors (whether detected visually or via detection device 1000) can then be compared with the colors indicted in Table 1, for example, for drug screening purposes.

The qualitative and subjective nature of the screening of the opioids by color change will be overcome by using smartphone spectrometer to read color changes quantitively after screening pad 500 is removed from the collection patch 100. As an example, one commercially available portable spectrometer (an exemplary detection device 1000) distributed by Allied Scientific Pro (Lighting Passport) weighs less than 80 grams and can be directly connected to a cell phone to perform color change analysis. Such a detection device 1000 is suitable for the screening of the agents (reacted indications 1050). The wavelength range is 380-780 nm which covers the visible light spectrum with 10 nm resolution is quite adequate for such a screening application. The spectrometer (detection device 1000) can be calibrated with known amounts of different agents. Opioid, reagent, and color information, such as that contained within Table 1, can be programmed into the smartphone and/or accessible by the smartphone so that all the screening results can appear on the smartphone without performing any intermediate data analysis.

Schedules I and II opioid substances which include heroin, fentanyl, morphine, oxycodone, and amphetamine from the list of candidate's agents have the highest potential for abuse and associated risk of fatal overdose due to respiratory depression. Screening of these five agents can be most important screening targets. Fentanyl can be abused and is subject to criminal diversion. Fentanyl and its analogues have rapid onset of symptoms and vary in duration of action, as they are 50-100 times more potent than morphine.

### Interstitial Body Fluid Transdermal Microneedles

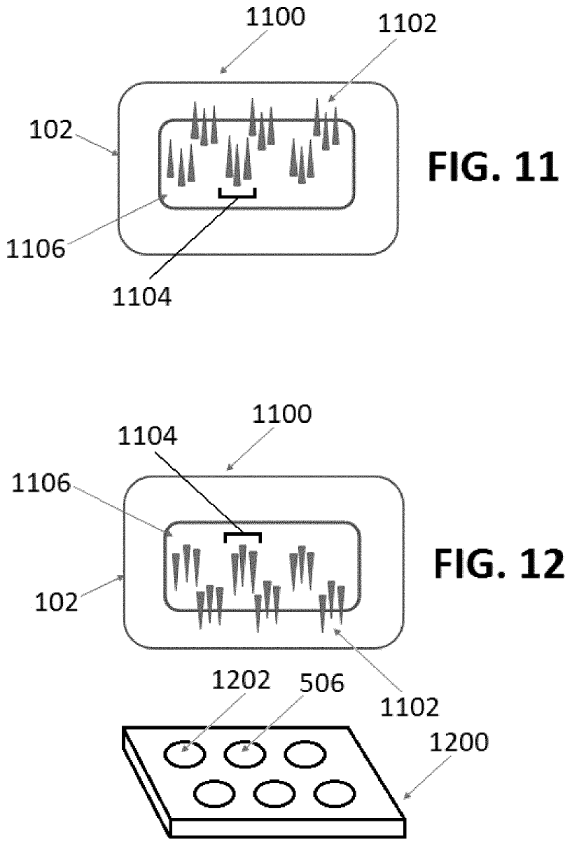

FIG. 11 shows an exemplary microneedle device 1100 of the present disclosure (a type of patch 100) incorporating a plurality of microneedles 1102. As shown in FIG. 11, microneedle device 1100 comprises a membrane 102 with an adhesive 106 positioned on at least part of the membrane 102. Adhesive 106, is used so to adhere microneedle device 1100 to the skin 402 of a wearer. Adhesive 106 can also be present between membrane 102 and a microneedle substrate 1106 to adhere microneedle substrate 1106 to membrane 102. So to protect and maintain sterility of microneedle device 1100, a release layer 108 can be used to cover the side of microneedle device 1100 having the plurality of microneedles, such as shown in FIG. 16. When release layer is removed, the plurality of microneedles 1102 are revealed, such as shown in FIG. 11.

As shown in FIG. 11, microneedles 1102 can be arranged upon microneedle substrate 1106 in microneedle groups 1104 as desired, whereby each microneedle group 1104 comprises a plurality of microneedles 1102. Said groups 1104 of microneedles 1102 can be arranged about microneedle substrate 1106 so to correspond with locations of wells 1202 defined within a corresponding reagent container 1200, whereby reagents 506 are present within said wells 1202 of reagent container 1200, such as shown in FIG. 12.

Microneedle devices 1100 of the present disclosure ideally include the fewest number of microneedles 1102 necessary in order to obtain a suitable sample of interstitial body liquid (IBL) from the skin 402 of the wearer of microneedle device 1100. For example, and as shown in FIG. 11, each group 1104 of microneedles 1102 contains three microneedles 1102, and with six groups 1104 (an exemplary number of groups containing an exemplary number of biocompatible reagents 506), that would be eighteen microneedles 1102 in total. Other microneedle devices 1100 may include any desired number of groups 1104 of microneedles 1102, with any desired number of microneedles 1102 per group 1104, such as a) six groups 1104 of three microneedles 1102 each (so eighteen total microneedles 1102), b) six groups 1104 of six microneedles 1102 each (so thirty-six total microneedles 1102), c) four groups 1104 of four microneedles 1102 each (so sixteen total microneedles 1102), d) six groups 1104 of twelve microneedles 1102 each (so seventy-two total microneedles 1102), etc. As referenced herein, six reagents 506 can be used to identify eleven different types of opioids, such as shown in FIG. 11, so exemplary and perhaps preferred microneedle device 1100 embodiments of the present disclosure would comprise six groups 1104 of microneedles 1102, each group 1104 corresponding ultimately to one reagent 506.

In some embodiments of microneedle devices 1100 of the present disclosure, microneedle devices 1100 comprises a microneedle substrate 1106 (which may the same as or similar to substrate 804), which is formed as part of an overall unit with microneedles 1102, or which is coupled to microneedles 1102 to help complete an embodiment of the microneedle device 1100 that can withstand the desired uses as referenced herein. Substrate 1106, as referenced herein, can be relatively flexible so to accommodate the irregular topography of the surface of the skin 402 due to macroscopic curvature of different body regions to prevent breakage of microneedles 1102 during insertion. As shown in FIG. 12, microneedle substrate 1106 can be adhered to membrane 102 on one side and microneedle substrate 1106 on another, using adhesive 106, as may be desired.

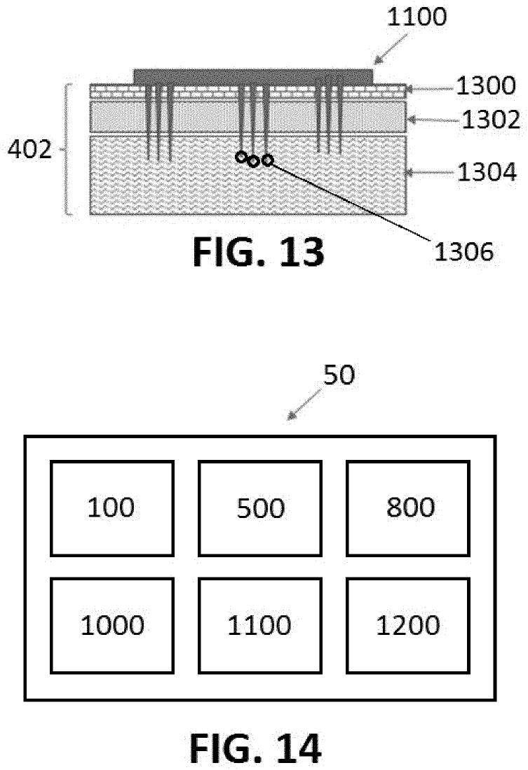

FIG. 13 shows an exemplary microneedle device used to extract IBL from the skin 402 so to at least partially coat the microneedles 1102 with IBL. FIG. 13 shows several layers of skin 402, including the stratum corneum 1300, viable epidermis 1302, and dermis 1304 containing IBL, from the outside moving inward. When microneedle device 1100 is positioned upon the skin 402 (first the stratum corneum 1300), microneedle device 1100 can then be pressed in the direction of skin 402 to cause microneedles 1102 to puncture the stratum corneum 1300, the viable epidermis 1302, and the dermis 1304, in that order, so that when completely positioned upon the skin 402, microneedle device 1100 contacts the skin 402, and the relative tips of microneedles 1102 are positioned within the dermis 1304. This allows IBL to at least partially coat microneedles 1102, so that when microneedle device 1100 is removed from the skin 402, IBL remains on said microneedles 1102.

Once microneedles 1102 are at least partially coated with IBL, said microneedles 1102 can be dipped into wells 1202 defined within a corresponding reagent container 1200, whereby reagents 506 are present within said wells 1202 of reagent container 1200, such as shown in FIG. 12. IBL present on said microneedles 1102 can react with reagents 506 within wells 1202 of reagent container 1200, causing color-changing reactions to occur should opioids or chemicals relating thereto be present upon said microneedles 1102. A detection device 1000, such as shown in FIG. 10, could be used to detect the colors within wells 1202 of reagent container 1200, or said colors could be detected visually should the colors be intense enough to detect visually.

FIG. 14 shows a block diagram of an exemplary system 50 of the present disclosure, whereby system 50 comprises two or more of the following: patch 100, screening pad 500, needle device 800, detection device 1000, microneedle device 1100, and/or reagent container 1200.

It is noted that metal and silicone microneedles (MNs) 1102 are not favored as the tip may break in the skin which will result in irritation. Silicon MNs 1102 require clean rooms, waste disposal issues, and their FDA approvals can be questionable, although some form of them has been approved. Open MNs 1102 are also not favored due to potential clogging in the opening of MN 1102 by tissue, thus preventing the entrance of the IBL; however, a solution to this problem is disclosed herein, as noted in further detail below. The hydrogel forming materials 1306, such as shown in FIG. 13, for the opioids screening application can be used, as referenced herein. The needle tips swell in skin to produce conduits. The opioids can diffuse from IBL of the dermal microcirculation using these conduits.

One candidate material 1306 can be aqueous blends containing 20% w/w Gantrez® AN-139 polymetric microneedles 1102. It is robust and not only punctures the stratum corneum 1300 of human skin in vivo, but also protrudes quite deeply into the underlying viable epidermis 1302 and upper dermis 1304 with relatively low insertion force of 0.03 N(newton)/MN. The height of said microneedles 1102 are or about 600 μm with about 500 μm extended into the skin. The interspacing of MN at the base is about 300 μm with the width at the base of about 300 μm. The MN can fabricated by laser based micro-molding technique. For example, an array of 11×11 needles (forming an exemplary needle device 800 and/or microneedle device 1100) with these dimensions takes about five minutes to be machined at ambient temperature using current technology. The baseplate (substrate 804 or 1204) can ideally possess some degree of flexibility to accommodate the irregular topography of the skin 402 surface due to macroscopic curvature of different body regions to prevent break of MN 1102 during insertion.

The following are two exemplary methods for screening opioids. In each option, an eighteen MNs 1102 array, a set of three MNs 1102 for each six reagents is utilized. The eighteen MN 1102 array can be configured in an area of 2 cm² with the dimensions indicated above. Smartphone spectrometer (an exemplary detection device 1000) can be used to read three color changes for each reagent and calculate the average. The schematic of the MN array is shown in FIG. 11 for the application namely the collection of IBL using a microneedle device 1100, and processing said microneedle device 1100 to identify one or more reacted indications 1050.

### Exemplary Method #1

An array of hydrogel material 1306 MN 1102 can be used where the polymer swells when absorbing the body fluid in the dermis layer. The MN 1102 array resides there for specified residence time. The reagents will be applied to MN 1102 once it is taken out to screen the opioids, such as by way of applying an exemplary reagent container 1200 of the present disclosure thereto.

### Exemplary Method #2

An array of hydrogel material 1306 MN 1102 can be used and coat it with the respective reagents before penetrating it to the dermis layer and then take out after specified residence time to detect the color change to screen the opioids. The reagents will have sufficient time to mix with the IBL during the process of hydrogel 1306 swelling. These reagents need to be biocompatible noting that although they tend to stay in the body for short time.

### Exemplary Method #3

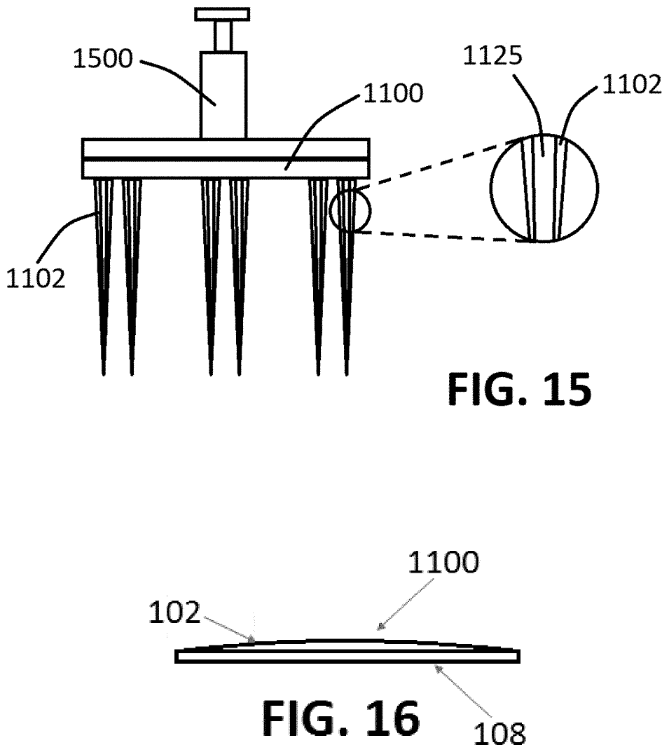

A microneedle device 1100 comprising an array (multiple groups 1104) of microneedles 1102 can be used as referenced herein, but also a) using hollow microneedles 1102 (microneedles 1102 having a channel or lumen 1125 defined therein, as best seen in the magnified inset shown in FIG. 15), and b) using a suction source/mechanism. Such a microneedle device 1100 is shown in FIG. 15, whereby microneedle device 1100 is coupled to, or is formed along with, a suction source/mechanism 1500, such as a syringe, vacuum source, and the like. Procedurally, microneedle device 1100 with hollow microneedles 1102 can be positioned upon the skin 402 as shown in FIG. 13, and while microneedles 1102 are positioned within the skin 402, suction from suction source/mechanism 1500 can cause IBL to flow within lumens 1125 of microneedles 1102, whereby the IBL from said lumens 1125 can be tested for opioids or chemicals relating thereto with reagents 506 as referenced herein.

Other methods, using various embodiments of patches 100, screening pads 500, needle devices 800, detection devices 1000, and/or microneedle devices 1100 of the present disclosure, can also be performed consistent with the present disclosure.

While various embodiments of systems, devices, and methods for using and manufacturing the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCE LIST

1. ANNUAL SURVEILLANCE REPORT OF DRUG-RELATED RISKS AND OUTCOMES, CDC National Center for Injury Prevention and Control, 2018.
2. WHO, http://www.who.int/substance_abuse/information-sheet/en, August 2018.
3. Kintz P, Brenneisen R, Bundelli P, Mangin P. Sweat testing for heroin and metabolites in a heroin maintenance program. Clinical Chemistry. 43(5), 736-739.1997.
4. Spiehler V, Fay J, Fogerson R, Schoendorfer D, Niedbala R S. Enzyme immunoassay validation for qualitative detection of cocaine in sweat. Clin. Chemistry, 42:34-8, 1996.
5. Cone E J. New developments in biological measures of drug prevalence. NIDA Research Monograph, 167,108-29,1997.
6. Caplan Y H, Goldberger B A. Alternative Specimens for Workplace Drug Testing. Journal of Analytical Toxicology, 25, 396-399, 2001.
7. Taylor J R, Watson I D, Tames F J, Lowe D. Detection of drug use in a methadone maintenance clinic: Sweat patches versus urine testing. Addiction, 93, 6, 847-853, 1998.
8. Kidwell D A, Smith F P., Susceptibility of PharmChek™ drugs of abuse patch to environmental contamination, Forensic Science International, 116, 89-106, 2001.
9. Marek C. Chawarski, D A Fiellin, P G O'Connor, M Bernard, and R S Schottenfeld, Utility of sweat patch testing for drug use monitoring in outpatient treatment for opiate dependence, J Subst. Abuse Treat, 33, 4, 411-415, 2007.
10. Donnelly, R. F. et al, Optimization and Characterization of Polymeric Microneedle Arrays Prepared by a Novel Laser-Based Micro-molding Technique, Pharm, 28, 41-57, 2011.
11. Larraneta, E., Lutton, R. E., Woolfson, A. D., Donnelly R. F., Microneedle arrays as transdermal and intradermal drug delivery systems: Materials science, manufacture and commercial development, Materials Science and Engineering R, 104,1-32, 2016.
12. Levisky J A, Bowerman D L, Jenkins W W, Karch S B. Drug deposition in adipose tissue and skin: Evidence for an alternative source of positive sweat patch tests, Forensic Science International, 110, 35-46, 2000.
13. Joseph R E Jr, Oyler J M, Wstadilc A T, Ohuoha C, Cone E J. Drug testing with alternative matrices 1. Pharmacological effects and disposition of cocaine and codeine in plasma, sebum, and stratum corneum. Journal of Analytical Toxicology, 22,1,6-17, 1998.
14. Willis I, Harris D R, Moretz W. Normal and abnormal variations in eccrine sweat gland distribution. Journal of Investigative Dermatology, 60,2,98-103, 1973.
15. Burns M, Baselt R C. Monitoring drug use with a sweat patch: an experiment with cocaine. Journal of Analytical Toxicology, 19, 41-8, 1995.
16. Cone E J, Hillgrove M J, Jenkins A J, Keenam R M, Darwin W D. Sweat testing for heroin, cocaine and metabolites. Journal of Analytical Toxicology, 19, 298-305, 1995.
17. Kintz P, Edel Y, Tracqui A, Mangin P. Sweat testing in opioid users with a sweat patch. Journal of Analytical Toxicology, 20, 393-7, 1996.
18. Kintz P, Tracqui A, Jamey C, Mangin P. Detection of codeine and phenobarbital in sweat collected with a sweat patch. Journal of Analytical Toxicology, 20:197-201. 1996.
19. Kintz P, Tracqui A, Mangin P. Sweat testing for benzodiazepines. Journal of Forensic Sciences, 41,851-4,1996.
20. Nuffield Foundation/Biosciences Federation, Downloaded from Practicalbiology.org, 2009.
21. Carl V. Gisolfi, Water Requirements During Exercise in the Heat, Institute of Medicine (US) Committee on Military Nutrition Research; Marriott B M, editor. Washington (DC): National Academies Press (US); 1993.
22. Pearson, J. Postmortem Toxicology Findings of Acetyl Fentanyl, Fentanyl, and Morphine in Heroin Fatalities in Tampa, Fla., Acad. Forensic Pathol., 5, 4, 676-689, 2015.
23. Kool J, et al. Suctionblister fluid as potential bodyfluid for biomarkerproteins. Proteomics 7, 3638-3650, 2007.
24. Müller A C, et al. A comparative proteomic study of human skin suction blister fluid from healthy individuals using immunodepletion and iTRAQ labeling. J Proteome Res 11, 3715-3727, 2012.
25. Skin anatomy. Available at https://emedicine.medscape.
26. Aukland K, Nicolaysen, Interstitial fluid volume: Local regulatory mechanisms. Physiol Rev 61, 556-643, 1981.
27. Samanta, P. P., and Prausnitza, M. R., Mechanisms of sampling interstitial fluid from skin using a microneedle patch, PNAS, 115, 2018.
28. Carol L O'Neal, Dennis J Crouch, Alim A Fatah, Validation of twelve chemical spot tests for the detection of drugs of abuse, 189-201, 2000.
29. Lighting passport smartphone spectrometer, https://www.lightingpassport.com, 2018.
30. “WCPI Focus on Pain Series: The Three Faces of Fentanyl” can be found: Caution-http://archive.li/V38XW <Caution-http://archive.li/V38XW> (Updated Sep. 26, 2018).
31. “Drug Facts: Fentanyl” National Institute on Drug Abuse, US National Institutes of Health. June 2016.
32. “Commission on Narcotic Drugs takes decisive step to help prevent deadly fentanyl overdoses” Commission on Narcotic Drugs, United Nations Office on Drugs and Crime, 16 Mar. 2017.

1. A microneedle device, comprising:
a membrane having an adhesive thereon; and
a microneedle substrate adhered to the membrane using the adhesive;
a plurality of microneedles; and
a release layer positioned upon the microneedle device to cover the microneedle substrate.

2. The microneedle device of claim 1, wherein the microneedle substrate has the plurality of microneedles coupled thereto.

3. The microneedle device of claim 1, wherein the microneedle substrate is formed as part of an overall unit with the plurality of microneedles.

4. The microneedle device of claim 1, wherein the plurality of microneedles comprises at least six groups of three microneedles.

5. The microneedle device of claim 1, wherein the plurality of microneedles are hydrogel forming microneedles.

6. The microneedle device of claim 1, forming part of a system, the system further comprising a reagent container having wells defined therein, the wells holding reagents, the reagents reactive with interstitial bodily fluid containing opioids or chemicals relating thereto.

7. The system of claim 6, wherein the plurality of microneedles are arranged about the microneedle substrate so as to correspond with the wells defined within the reagent container.

8. The system of claim 7 wherein the plurality of microneedles comprises multiple groups of microneedles, and each group corresponds to a well.

9. The system of claim 7 wherein the reagent container has at least two wells, each well holding a different reagent that reacts with interstitial body fluid to detect a different opioid or chemical relating thereto.

10. A method of using a microneedle device to detect opioids and other drugs, comprising the steps of:
placing a microneedle device of the present disclosure upon skin of a wearer so as to cause at least part of a plurality of microneedles of the microneedle device to enter a dermis of the skin; and
removing the microneedle device from the skin after a period of time elapses, said period of time being enough time to permit interstitial body fluid to at least partially coat or enter the plurality of microneedles.

11. The method of claim 10, further comprising the step of:
positioning the plurality of microneedles of the microneedle device into a wells of a reagent container to cause one or more reactions between the interstitial body fluid at least partially coating or entering the plurality of microneedles and at least one reagent within the wells of the reagent container, said one or more reactions resulting in one or more color changes, the one or more color changes indicative of the presence of one or more opioids and/or chemicals related thereto.

12. The method of claim 10, further comprising the step of:
prior to the step of placing the microneedle device upon the skin of the wearer, at least partially coating the plurality of microneedles with reagents; and
after removing the microneedle device from the skin, inspecting the plurality of microneedles in attempt to identify one or more color changes thereon, the one or more color changes indicative of the presence of one or more opioids and/or chemicals related thereto.

13. The method of claim 10, wherein the plurality of microneedles have lumens defined therethrough, wherein the step of placing the microneedle device upon the skin of the wearer further includes operating a suction device/mechanism coupled to or formed as part of the microneedle device to cause the interstitial body fluid to flow into the lumens of the plurality of microneedles.

14. The method of claim 13, further comprising the step of:
combining the interstitial body fluid with a plurality of reagents so to potentially cause one or more reactions between the interstitial body fluid and the reagents, said one or more reactions resulting in one or more color changes, the one or more color changes indicative of the presence of one or more opioids and/or chemicals related thereto.

15. The method of claim 10, wherein the step of placing microneedle device of the present disclosure upon the skin of a wearer causes a relative tip of the plurality of microneedles to rest within the dermis.

16. A method of screening for opioids and other drugs comprising the steps of:
placing a patch upon a skin of a wearer, the patch comprising:
a sweat-absorbent swatch and an adhesive layer; and
removing the patch from the skin after a period of time elapses, said period of time being enough time to permit sweat to transfer from the skin to the sweat-absorbent swatch; and
positioning a screening pad comprising one or more reagents upon the sweat-absorbent swatch of the patch such that the sweat and reagents chemically react resulting in one or more color changes, the one or more color changes indicative of the presence of one or more opioids and/or chemicals related thereto.

17. The method of claim 16 further comprising the step of:
after positioning the screening pad upon the sweat-absorbent patch, heating the sweat absorbent patch slightly to evaporate the residual liquid to increase agent concentrations.

18. The method of claim 16 further comprising the step of:
after positioning the screening pad upon a sweat-absorbent swatch of the patch, puncturing at least two blisters disposed on the screening pad, each of the at least two blisters containing at least one reagent that flows onto a sweat collection part of the patch to detect at least two opioids or related chemicals.

19. The method of claim 18 wherein a needle device is used to puncture the blisters one or more at a time or all at once so as to let the reagents flow and react with the sweat.

20. The method of claim 16 further comprising the step of using a detection device to determine the color changes.
